(19) United States
(12) Patent Application Publication
(10) Pub. No.: US 2007/0134691 A1
(43) Pub. Date: Jun. 14, 2007

(54) METHODS AND COMPOSITIONS FOR DETERMINING WHETHER A SUBJECT CARRIES A GENE MUTATION ASSOCIATED WITH HEREDITARY HEARING LOSS

(76) Inventors: IRIS SCHRIJVER, San Jose, CA (US); PHYLLIS GARDNER, Stanford, CA (US); ENELI OITMAA, Tartumae (EE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/559,575

(22) Filed: Nov. 14, 2006

Related U.S. Application Data
(60) Provisional application No. 60/738,713, filed on Nov. 21, 2005.

(51) Int. Cl. C12Q 1/68

(52) U.S. Cl. 435/6

(57) ABSTRACT

Methods are provided for determining whether a subject carries a gene mutation associated with Hereditary Hearing Loss (HHL). In practicing the subject methods, an array comprising a plurality of HHL-associated gene mutation probes is contacted with a nucleic acid sample from the subject, and the presence of any resultant surface bound target nucleic acids is detected to determine whether the subject carries a HHL-associated gene mutation. In addition, reagents and kits thereof that find use in practicing the subject methods are provided.

### CROSS REFERENCE TO RELATED APPLICATIONS

Applicant claims the benefit under 35 U.S.C. §119(e) of prior U.S. provisional application Ser. No. 60/738,713 filed Nov. 21, 2005, the disclosure of which is incorporated herein by reference.

### BACKGROUND

One third of the approximately 2 million deaf people in the United States have an inherited form of hearing loss. The rate of newborns with hearing loss is 1 in 1000 births, with approximately 60% due to a genetic cause. Hearing loss is subclassified by several criteria, including: type (conductive, sensorineural, central auditory dysfunction or mixed); cause (hereditary, including autosomal dominant, autosomal recessive, X-linked, and mitochondriaI, versus environmental); and association (syndromic or nonsyndromic). Syndromic deafness is associated with other physical defects, such as retinitis pigmentosa, euthymic goiter, craniofacial abnormalities, pigmentation, branchial cleft cysts or fistulae and renal disease, and long QT. By contrast, nonsyndromic deafness is not associated with other physical defects in a clear pattern.

Increased requirement for molecular genetic testing for HHL has been mandated both by the advent of newborn hearing screening programs in many states coupled with the identification of the many genes that cause specific forms of hearing loss. The advantages of molecular genetic testing in HHL, as supplementation to clinical diagnostic testing by means of audiometry, auditory brainstem response testing, evoked otoacoustic emissions and immittance testing, are compelling. Molecular genetic testing enables informed genetic counseling with respect to mode of inheritance, precise definition of etiology, and risk assessment. It can also facilitate early detection and intervention with treatment options (audiologic, linguistic, or opthamologic) that have a very important impact on the cognitive development of the patient, particularly those with prelingual or very early onset hearing loss. It can identify environmental risk factors, e.g., aminoglycosides in individuals with 12SrRNA mutations or middle ear associate syndromic features (e.g., long QT associated risk of syncope and sudden death in patients with Jervell and Lange-Nielsen syndromes). As clinical testing is increasingly implemented and as knowledge grows about the disease causing mutations in the varied genes that cause HHL, the demand for molecular genetic testing for HHL will inevitably increase.

### SUMMARY OF THE INVENTION

Methods are provided for determining whether a subject carries a gene mutation associated with Hereditary Hearing Loss (HHL). In practicing the subject methods, an array comprising a plurality of HHL-associated gene mutation probes is contacted with a nucleic acid sample from the subject, and the presence of any resultant surface bound duplex nucleic acids is detected to determine whether the subject carries a gene mutation associated with HHL. In addition, reagents and kits thereof that find use in practicing the subject methods are provided.

### BRIEF DESCRIPTION OF THE FIGURES

FIG. 1. Schematic depiction of the APEX development and process. The steps for development of the SNHL APEX array are demonstrated. For those steps in which gDNA was used, individual times are listed in order to reflect the time to assay completion in a research or clinical setting. (h=hours, min=minutes). Hands-on time of the assay is approximately 1 hour and 25 minutes.

FIG. 2. Examples of APEX mutation detection of a missense mutation (R143W) and a deletion (35delG) in the GJB2 gene.

a) R143W. For a normal allele, a signal for the sense (S) oligo is expected in the C channel and for the antisense (AS) oligo in the G channel (C/G). For a mutant allele a signal is expected for the sense oligo in the T channel and for the antisense oligo in the A channel (T/A). Row 1. Normal genotype for position R143. The normal C allele is present in S. In AS the normal G allele is detected (C/G). Row 2. Heterozygous for R143W. The normal C and mutant T alleles are detected in S. In AS, the normal G allele and the mutant A allele are present (CT/GA). Row 3. Negative control (no DNA added to the APEX reaction).

b) 35delG. For a normal allele, a signal for the sense oligo is expected in the G channel and for the antisense oligo in the C channel (G/C). For a mutant allele, a signal for the sense oligo is expected in the T channel and for the antisense oligo in the A channel (T/A). Row 1. Heterozygous for 35delG. In S the normal G allele and the mutant T allele are detected. In AS the normal C allele and the mutant A allele are identified (GT/CA). Row 2. Homozygous for 35delG. Row 3. Normal genotype for nucleotide position 35. Row 4. Negative control.

### DETAILED DESCRIPTION

Methods are provided for determining whether a subject carries a gene mutation associated with HHL. In practicing the subject methods, an array comprising a plurality of HHL-associated gene mutation probes is contacted with a nucleic acid sample from the subject, and the presence of any resultant surface bound target nucleic acids is detected to determine whether the subject carries a HHL-associated gene mutation. In addition, reagents and kits thereof that find use in practicing the subject methods are provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, an”, and “the” include plural referents unless the context clearly dictates otherwise. It is further noted that the claims may be drafted to exclude any optional element. As such, this statement is intended to serve as antecedent basis for use of such exclusive terminology as “solely, “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, the subject invention is directed to methods of determining whether a subject carries a HHL gene mutation, as well as compositions of matter and kits thereof that find use in practicing the subject methods. In further describing the invention, the subject methods are described first in greater detail, followed by a review of representative applications in which the methods find use, as well as reagents and kits that find use in practicing the subject methods.

Methods

The subject invention provides methods of determining whether a patient or subject carries a gene mutation associated with HHL. By “associated with HHL” is meant that the gene mutation has been linked or associated with HHL, i.e., the gene mutation has been observed in patients that have HHL and is positively correlated with the presence of HHL in the patient. By “carries” is meant that a subject has a gene mutation associated with HHL, where the subject may be heterozygous or homozygous for the particular mutation and be considered to carry the mutation. By HHL-associated gene mutation is meant a mutation in a gene associated with HHL, where representative genes associated with HHL include, but are not limited to, those listed in Table 1. The HHL-associated gene mutations that may be detected according to the subject invention may be deletion mutations, insertion mutations or point mutations, including substitution mutations. Representative specific HHL-associated gene mutations of interest include, but are not limited to, those mutations listed in Table 1.

TABLE 1
Representative HHL-Associated Gene Mutations.

| | |
|---|---|
| Amino acid change | Nucleotide change |

GJB2 (connexin 26) non-syndromic, dominant

| | |
|---|---|
| delE42 | 125delAGG |
| W44S | 131G > C |
| W44C | 132G > C |
| R75Q | 224G > A |
| M163L | 487A > C |
| D179N | 535G > A |
| R184Q | 551G > A |
| C202F | 605G > T |

GJB2 (connexin 26) non-syndromic, recessive

| | |
|---|---|
| na | −3172G > A |
| na | −3170G > A |
| M1V (p.0) | 1A > G |
| T8M | 23C > T |
| frameshift | 31del14 |
| frameshift | 31del38 |
| G12V | G to T at 35 |
| frameshift | 35delG |
| frameshift | 35insG |
| K15T | 44A > C |
| frameshift | 51del12insA |
| S19T | 56G > C |
| I20T | 59T > C |
| W24X | 71G > A |
| V27I + E114G | 79G > A + 341A > G |
| R32C | 94C > T |
| R32H | 95G > A |
| R32L | 95G > T |
| M34T | 101T > C |
| V37I | 109G > A |
| A40E | 119C > A |
| A40G | 119C > G |
| W44X | 132G > A |
| G45E | G > A134 |
| E47X | 139G > T |
| E47K | 139G > A |
| frameshift | 167delT |
| Q57X | 169C > T |
| frameshift | 176-191del16 |
| C64X | 192C > A |
| Y65X | 195C > G |
| Y65X | 195C > A |
| W77R | 229T > C |
| W77X | 231G > A |
| frameshift | 235delC |
| L79P | 236T > C |
| Q80X | 238C > T |
| Q80P | 239A > C |
| Q80R | 239A > G |
| I82M | 246C > G |
| V84L | 250G > C |
| S85P | 253T > C |
| A88S | 262G > T |
| L90V | 268C > G |
| L90P | 269T > C |
| frameshift | 269insT |
| M93I | 279G > A |
| V95M | G > A283 |
| Y97X | 291C > A or 291C > G |
| frameshift | 290-291insA |
| H100Y | 298C > T |
| frameshift | 299-300delAT |
| H100L | 299A > T |
| Del K102 | 302del3 (AGA) |
| E101G | 302A > G |
| frameshift | 310del14 |
| frameshift | 312del14 |
| frameshift | 314del14 |
| frameshift | 333-334delAA |
| S113R | 339T > G |
| delE120 | 360del3 (GAG) |
| K122I | 365A > T |
| Q124X | 370C > T |
| R127H | 380G > A |
| W133X | 398G > A |
| Y136X | 408C > A |
| S139N | 416G > A |
| R143W | 427C > T |
| E147K | 439G > A |
| E147X | 439G > T |
| frameshift | 486insT |
| R165W | 493C > T |
| 486insT | 504insAAGG |
| frameshift | 509del14 |
| frameshift | 509insA |
| frameshift | 515del17 |
| W172X | 516G > A |
| C174R | 520T > C |
| P175T | 523C > T |
| V178A | 533T > C |
| R184W | 550C > G |
| R184P | 551G > C |
| frameshift | 572delT |
| S198F | 596C > T |
| frameshift | 605ins46 |
| I203K | 608TC > AA |
| N206S | 617A > G |
| frameshift | 631delGT |
| L214P | 641T > C |
| frameshift | 645-648delTAGA |

GJB3 (connexin 31) non-syndromic, dominant

| | |
|---|---|
| R180X | 538C > T |
| E183K | 547G > A |
| Del Ile141 | 421del3 (ATT) |
| I141V | 421A > G) |
| P223T | 667C > A |

GJB6 (connexin 30) non-syndromic, dominant

| | |
|---|---|
| T5M | 14C > T |
| frameshift | 63delG |

GJB6 (connexin 30) non-syndromic, recessive

| | |
|---|---|
| na | ˜309 kb del |

GJA1 (connexin 43) non-syndromic, recessive

| | |
|---|---|
| L11F | 31C > T |
| V24A | 71T > C |

Mitochondrial non-syndromic

| | |
|---|---|
| 12SrRNA | 1555A > G |
| tRNA Ser | 7445A > G |
| tRNA Ser | 7472insC |
| tRNA Ser | 7511T > C |

SLC26A4 (Prestin), non-syndromic, recessive

| | |
|---|---|
| na | IVS2 − 2A > G |

SLC26A4 (Pendrin) Pendred syndrome, recessive

| | |
|---|---|
| na | −3 − 2A > G |
| M1T | 2T > C |
| R24G | 70C > G |
| S28R | 84C > A |
| E29Q | 85G > C |
| na | 165 − 1 G > A (IVS2 − 1G > A) |
| Y78C | 233A > G |
| frameshift | 279delT |
| A104V | 311C > T |
| Y105C | 314A > G |
| A106D | 317C > A |
| L117F | 349C > T |
| frameshift | 336_377insT |
| T132I | 395C > T |
| S133T | 397T > A) |
| frameshift | 407_411delTCTCA |
| V138F | 412G > T |
| frameshift | 25bpdel + 5 bp ins |
| frameshift | 415 + 7 A > G (IVS4 + 7A > G) |
| G139A | 416G > C |
| T193I | 578C > T |
| G209V | 626G > T |
| L236P | 707T > C |
| frameshift | 753_756delCTCT |
| frameshift | 783_784insT |
| D271H | 811G > C |
| frameshift | 917delT |
| na | 766 − 2 A > G (IVS7 − 2A > G) |
| na | 918 + 1 G > A (IVS7 + 1G > A) |
| na | 1001 + 1G > A (IVS8 + 1G > A) |
| na | 919 − 2A > G (IVS8 − 2A > G) |
| N324Y | 970 A > T |
| F335L | 1003T > C |
| K369E | 1105A > G |
| A372V | 1115C > T |
| frameshift | 1147delC |
| E384G | 1151A > G |
| S394del | 1181del3 (TCT) |
| frameshift | 1197delT |
| R409H | 1226G > A |
| T410M | 1229C > T |
| A411P | 1231G > C |
| T416P | 1246A > C |
| Q421R | 1262A > G |
| na | 1264 − 1 G > C |
| del A429 | 1284del3 (TGC) |
| L445W | 1334T > G |
| frameshift | 1334_1335insAGTC |
| Q446R | 1337 A > G |
| frameshift | 1341delG |
| V480D | 1440T > A |
| I490L | 1468A > C |
| G497S | 1489G > A |
| T508N | 1523C > A |
| frameshift | 1536_1537delAG |
| na | IVS13 + 9C > G |
| Y530H | 1588T > C |
| na | 1614 + 1G > A (IVS14 + 1G > A) |
| Y556H | 1666T > C |
| Y556C | 1667A > G |
| C565Y | 1694G > A |
| L597S | 1790T > C |
| V609G | 1826T > G |
| frameshift | 1898delA |
| V653A | 1958T > C |
| F667C | 2000T > G |
| G672E | 2015G > A |
| F683S | 2048T > C |
| frameshift | 2111_2112insGCTGG |
| frameshift | 2127delT |
| T721M | 2162C > T |
| H723R | 2168A > G |
| D724G | 21612A > G |
| frameshift | 2182_2183insG |
| G740S | 2217G > A |
| X781W | 2343A > G |

GJB2 (connexin 26) syndromic, dominant

| | |
|---|---|
| G12R | 34G > C |
| S17F | 50C > T |
| D50N | 148G > A |
| N54K | 162C > A or 162C > G |
| G59A | 176G > C |
| D66H | 196G > C |
| R75W | 223C > T |
| R75Q | 224G > A |

na = not applicaple

In practicing the methods of the subject invention, a host or subject is simultaneously screened for the presence of a plurality of different HHL-associated gene mutations. In certain embodiments, the host or subject is simultaneously screened for the presence of at least 25 different mutations, usually at least about 40 different gene mutations and often at least about 50 different gene mutations. In certain other embodiments the number of different gene mutations that are simultaneously screened is at least about 75, at least about 100, at least about 150, at least about 175, at least about 200 or more. In certain embodiments, the HHL gene mutations that are screened are from two or more different HHL-associated genes, e.g., about 3 or more, about 4 or more, about 5 or more, about 10 or more, about 15 or more, about 25 or more, etc. In certain embodiments, the HHL-associated genes that are screened are genes that are associated with both syndromic and non-syndromic causes of HHL. In certain embodiments, the HHL-associated gene mutations that are screened or assayed in a given test include at least about 25 of the mutations listed in Table 1, such as at least about 50 of the mutations listed in Table 1, including at least about 75, at least about 100, at least about 125, at least about 150, at least about 175, at least about 200 or more, including all, of the mutations listed in Table 1.

In certain embodiments of the present invention, the host may be simultaneously screened for the presence of a plurality of HHL-associated gene mutations using any convenient protocol, so long as at least about 30, and typically at least about 50, of the mutations appearing in Table 1 are assayed. In such embodiments, representative protocols for screening a host for the presence of the HHL gene mutations include, but are not limited to, array-based protocols, including those described in U.S. Pat. Nos. 6,027,880 and 5,981,178, the disclosures of which are herein incorporated by reference.

In certain embodiments of interest, an arrayed primer extension assay protocol (e.g., as described in Kurg et al., Genet. Test (2000) 4:1-7 and Tonisson et al., Microarray Biochip Technology (ed. Schena, Eaton Publishing, Natick, Mass.) (2000) pp. 247-263) is employed to screen a subject for the presence of a plurality of different HHL gene mutations. In such embodiments, an array of a plurality of distinct HHL-associated gene mutation specific probes is first contacted with a nucleic acid sample from the host or subject. The resultant sample-contacted array is then subjected to primer extension reaction conditions in the presence of two or more, including four, distinguishably labeled dideoxynucleotides. The resultant surface bound labeled extended primers are then detected to determine the presence of at least one HHL-associated gene mutation in the host or subject from which the sample was obtained. Each of these steps is now described in greater detail below.

As summarized above, the first step of the protocol employed in these embodiments is to contact an array of a plurality of HHL-associated gene mutation probes with a nucleic acid sample from the host or subject being screened. The array employed in these embodiments includes a plurality of HHL-associated gene mutation probes immobilized on a surface of a solid substrate, where each given probe of the plurality is immobilized on the substrate surface at a known location, such that the location of a given probe can be used to identify the sequence or identity of that probe. Each given probe of the plurality is typically a single stranded nucleic acid, having a length of from about 10 to about 100 nt, including from about 15 to about 50 nt, e.g., from about 20 to about 30 nt, such as 25 nt. The arrays employed in the subject methods may vary with respect to configuration, e.g., shape of the substrate, composition of the substrate, arrangement of probes across the surface of the substrate, etc., as is known in the art. Numerous array configurations are known to those of skill in the art, and may be employed in the subject invention. Representative array configurations of interest include, but are not limited to, those described in U.S. Pat. Nos.: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

As mentioned above, a feature of the arrays employed in this embodiment of the invention is that they include a plurality of different HHL-associated gene mutation probes. The total number of HHL-associated gene mutation probes that may be present on the surface of the array, i.e., the total number of HHL-associated gene mutations that may be represented on the array, may vary, but is in many embodiments at least about 25 or more, usually at least about 40 or more and often at least about 50 or more different gene mutations, where in many embodiments the number of different gene mutations that are represented on the array is at least about 75, at least about 100, at least about 150, at least about 175, at least about 200 or more. In certain embodiments, the HHL-associated gene mutations that are represented on the array in the form of probes include at least about 25 of the mutations listed in Table 1, such as at least about 50 of the mutations listed in Table 1, including at least about 75, at least about 100, at least about 125, at least about 1507 at least about 175, at least about 200 or more, including of all of, the mutations listed in Table 1. In certain embodiments, the arrays employed in the subject methods include a pair of different probes for each given HHL-associated gene mutation represented on the array. Typically, the pair of probes corresponds to the sense and antisense strand of the HHL-associated gene region that includes the mutation of interest. Such a configuration is known in the art and described in Kurg et al., Genet. Test (2000) 4:1-7.

As summarized above, the first step in the subject methods is to contact a nucleic acid sample obtained from the host or subject being screened with the array to produce a sample contacted array. The nucleic acid sample is, in certain embodiments, one that contains an amplified amount of fragmented HHL-associated gene nucleic acids, e.g., DNA or RNA, where in certain other embodiments the nucleic acid sample is a DNA sample. The nucleic acid sample is typically prepared from one or more cells or tissue harvested from a subject to be screened using standard protocols. Following harvesting of the initial nucleic acid sample, the sample is subjected to conditions that produce amplified amounts of one or more of the HHL-associated genes present in the sample which are to be probed on the array. While any convenient protocol may be employed, in certain embodiments the sample is contacted with a pair of primers that flank each region of interest of the HHL-associated gene, i.e., a pair of primers for each region of interest of each of the HHL-associated genes to be assayed, and then subjected to PCR conditions. This step results in the production of an amplified amount of nucleic acid for each particular region or location of the one or more HHL-associated genes of interest. Amplification protocols that find use in such methods are well known to those of skill in the art.

The resultant nucleic acid composition that includes an amplified amount of the HHL-associated gene sequences is then fragmented to produce a fragmented HHL-associated gene sample. Fragmentation may be accomplished using any convenient protocol, where representative protocols of interest include both physical (e.g., shearing) and enzymatic protocols. In certain embodiments, an enzymatic fragmentation protocol is employed, where the nucleic acid sample is contacted with one or more restriction endonucleases that cleave the one or more HHL-associated gene nucleic acids into two or more fragments.

The resultant amplified fragmented HHL-associated gene nucleic acid sample is then contacted with the array under conditions sufficient to produce surface immobilized duplex nucleic acids between host or subject derived nucleic acids and any complementary probes present on the surface of the array. In certain embodiments, the sample is contacted with the array under stringent hybridization conditions. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in IxSSC at 45° C. Alternatively, hybridization to filter-bound DNA in 0.5 M NaHPO₄, 7% sodium dodecyl sulfate (SDS), 1 mM EDTA at 65° C., and washing in 0.1 ×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Put another way, stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Sample contact and washing of the array as described above results in the production of a sample contacted array, where the sample contacted array is characterized by the presence of surface bound duplex nucleic acids, generally at each position of the array where probe nucleic acids and target nucleic acids in the sample have sufficiently complementary sequences to hybridize with each other into duplex nucleic acids under the conditions of contact, e.g., stringent hybridization conditions.

Following production of the sample contacted array, as described above, the presence of any HHL-associated gene mutations in the assayed nucleic acid sample, and therefore the host genome from which the sample was prepared, is detected. Depending on the nature of the array employed and the detection protocol used, a number of different protocols may be employed for determining the presence of one or more HHL-associated gene mutations in the assayed nucleic acid sample. For example, in certain embodiments in which the array includes immobilized probes that specifically bind only to target nucleic acids generated from mutated genomic sequences, detection of surface bound duplex nucleic acids can be used directly to determine the presence of one or more HHL-associated gene mutations in the sample.

In certain embodiments, the presence of any HHL-associated gene mutations is detected using a primer extension protocol, in which the surface bound probe component of the duplex nucleic acid acts as a primer which is extended in a template dependent primer extension reaction using the hybridized complement of the probe which is obtained from the patient derived nucleic acid sample as a template. In these embodiments, the sample-contacted array is contacted with primer extension reagents and maintained under primer extension conditions.

Primer extension reactions are well known to those of skill in the art. In this step of the subject methods, the sample-contacted array is contacted with a DNA polymerase under primer extension conditions sufficient to produce the desired primer extension molecules. DNA polymerases of interest include, but are not limited to, polymerases derived from E. coli, thermophilic bacteria, archaebacteria, phage, yeasts, Neurosporas, Drosophilas, primates and rodents. The DNA polymerase extends the probe “primer” according to the template to which it is hybridized in the presence of additional reagents which may include, but are not limited to: dNTPs; monovalent and divalent cations, e.g. KCl, MgCl₂; sulfhydryl reagents, e.g. dithiothreitol; and buffering agents, e.g. Tris-Cl.

In certain embodiments, the primer extension reaction of this step of the subject methods is carried out in the presence of at least two distinguishably labeled dideoxynucleotide triphosphates, or ddNTPs. In certain of these embodiments, the primer extension reaction of this step of the subject methods is carried out in the presence of at least four distinguishably labeled dideoxynucleotide triphosphates (ddNTPs), e.g., ddATP, ddCTP, ddGTP and ddTTP, and in the absence of deoxynucleotide triphosphates (dNTPs).

Extension products that are produced as described above are typically labeled in the present methods. As such, the reagents employed in the subject primer extension reactions typically include a labeling reagent, where the labeling reagent is typically a labeled nucleotide, which may be labeled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In certain embodiments, the label is a directly detectable label, such as a fluorescent label, where the labeling reagent employed in such embodiments is a fluorescently tagged nucleotide(s), e.g., ddCTP. Fluorescent moieties which may be used to tag nucleotides for producing labeled probe nucleic acids include, but are not limited to; fluorescein, the cyanine dyes, such as Cy3, Cy5, Alexa 555, Bodipy 630/650, and the like. Other labels may also be employed as are known in the art.

In the primer extension reactions employed in the subject methods of these embodiments, the surface of the sample contacted array is maintained in a reaction mixture that includes the above-discussed reagents at a sufficient temperature and for a sufficient period of time to produce the desired labeled probe “primer” extension products. Typically, this incubation temperature ranges from about 20° C. to about 75° C., usually from about 37° C. to about 65° C. The incubation time typically ranges from about 5 min to about 18 hr, usually from about 1 hr to about 12 hr.

Primer extension of any duplexes on the surface of the array substrate as described above results, in certain embodiments, in the production of labeled primer extension products. In those embodiments where primer extension is carried out solely in the presence of distinguishably labeled ddNTPs, as described above, the primer extension reaction results in extension of the probe “templates” by one labeled nucleotide only.

Following production of labeled primer extension products, as described above, the presence of any labeled products is then detected, either qualitatively or quantitatively. Any convenient detection protocol may be employed, where the particular protocol that is used will necessarily depend on the particular array assay, e.g., the nature of the label employed. Representative detection protocols of interest include, but are not limited to, those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97110365; WO 97/27317; EP 373 203; and EP 785 280.

Where the primer extension products are fluorescently labeled primer extension products, any convenient fluorescently labeled primer extension protocol may be employed. In certain embodiments, a “scanner” is employed that is capable of scanning a surface of an array to detect the presence of labeled nucleic acids thereon. Representative scanner devices include, but are not limited to, those described in U.S. Pat. Nos. 5,585,639; 5,760,951; 5,763,870; 6,084, 991; 6,222,664; 6,284,465; 6,329,196; 6,371,370 and 6,406,849. In certain embodiments, the scanner employed is one that is capable of scanning an array for the presence of four different fluorescent labels, e.g., a four-channel scanner, such as the one disclosed in published U.S. Patent Application Serial No. 20010003043; the disclosure of which is herein incorporated by reference.

The final step in these embodiments of the subject methods is to determine the presence of any HHL-associated gene mutations in the assayed sample, and therefore the host from which the sample was obtained, based on the results of the above surface immobilized duplex nucleic acid detection step. In this step of the subject methods, any detected labeled duplex nucleic acids, and specifically labeled extended primers, are employed to determine the presence of one or more HHL-associated gene mutations in the host from which the screened sample was obtained. This step is practiced by simply identifying the location on the array of the labeled duplex, and then identifying the probe(s) (and typically sequence thereof) of the probe “primer” at that location which was extended and labeled. Identification of the probe(s) provides the specific HHL-associated gene mutation(s) that is present in the host from which the sample was obtained.

Using the above described protocols, the presence of one or more HHL-associated gene mutations in the genome of a given subject or host may be determined. In other words, whether or not a host carries one or more HHL-associated gene mutations may be determined using the subject methods. The subject methods may be employed to determine whether a host is homozygous or heterozygous for one or more HHL gene mutations. A feature of the subject methods is that they provide for a highly sensitive assay for the presence of HHL gene mutations across a broad population. For example, they provide for a sensitivity of at least about 60%, including at least about 65%, 70%, 75% or higher, e.g., 80%, 85%, 90% or higher, in a plurality of different racial backgrounds, including Caucasian, Asian, Hispanic and African racial backgrounds.

In certain embodiments, the methods of the present application are used to detect the presence of one or more HHL-associated gene mutations in multiple subjects with a high degree of accuracy. By high degree of accuracy is meant that at least about 90% of the HHL-associated gene mutations present in the samples are accurately identified using the methods of the invention, including accuracies of about 92%, about 95%, about 97%, about 99% and up to an accuracy of 100%. In these embodiments, multiple samples from different subjects are be processed according to the methods of the present invention with a single APEX array being used for each individual subjects sample (e.g., in a high throughput fashion). The number of distinct samples processed in these embodiments may vary widely, including, but not limited to, 2 samples or more, 5 samples or more, 20 samples or more or up to 100 samples or more, where the multiple samples are all evaluated with the high degree of accuracy, as reviewed above. In general, the limitation in the number of samples that can be processed at a time is based on the resources of one employing the methods of the present invention. As such, no limitation in this regard is intended.

Utility

The subject methods find use in a variety of different applications. In certain embodiments, the above-obtained information is employed to diagnose a host, subject or patient with respect to whether or not they carry a particular HHL-associated gene mutation.

In certain other embodiments, the subject methods are employed to screen potential parents to determine whether they risk producing offspring that are homozygous for one or more HHL-associated mutations. In other words, the subject methods find using in genetic counseling applications, where prospective parents can be screened to determine there potential risk in producing a child that is homozygous for a HHL-associated gene mutation (or heterozygous for two disease causing mutations) and will suffer from a disease associated therewith, e.g., hearing loss.

In certain other embodiments, the subject methods and compositions are employed to screen populations of individuals, e.g., to determine frequency of various mutations. For example, a select population of individuals, e.g., grouped together based on race, geographic region, etc., may be screened according to the subject invention to identify those mutations that appear in members of the population and/or determine the frequency at which such identified mutations appear in the population.

Reagents and Kits

Also provided are reagents and kits thereof for practicing one or more of the above-described methods. The subject reagents and kits thereof may vary greatly depending on the particular embodiment of the invention to be practiced. Reagents of interest include, but are not limited to: nucleic acid arrays (as described above); HHL-associated gene specific primers, e.g., for using in nucleic acid sample preparation, as described above, one or more uniquely labeled ddNTPs, DNA polymerases, various buffer mediums, e.g. hybridization and washing buffers, and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

Materials and Methods

Mutation Selection

The 198 mutations on the APEX microarray were selected and characterized from multiple sources including the 1) Connexin Deafness home page [website (http://) followed by (davinci.org) followed by (.es/deafness/)], 2) mitochondrial mutation literature and the Mitomap database [Fischel-Ghodsian N., Mitochondrial deafness. Ear Hear 2003;24:303-313; and website (http://www) followed by (.mitomap.org)], 3) Hereditary Hearing Loss home page [website (http://) followed by (webhost.ua) followed by (.ac.be/hhh/)], and 4) Human Gene Mutation Database [website (http://www) followed by (.hgmd.cf.ac.ukt)]. The mutations of interest are listed in Table 1.

Oligonucleotide Microchips

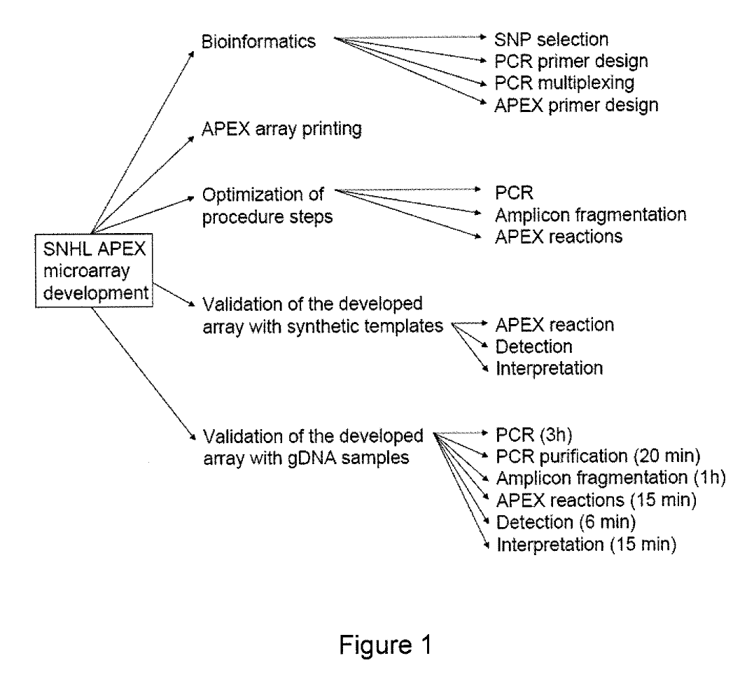

An overview of the development of the SNHL APEX array is presented in FIG. 1. The appropriate wild-type gene sequences for both the sense and antisense directions [website (http://www) followed by (.ncbi.nlm.nih) followed by (.gov/Genbank/)] were used as templates for oligonucleotide primer design. The 25 bp oligonucleotides have 6-carbon amino linkers at their 5′ end and are obtained from MWG (Munich, Germany). Most of these oligonucleotides were designed to extend by 1 bp in the wild-type and mutant sequence, except when deletions or insertions are present that have the same nucleotide in the 1 bp direction as the expected wild type sequence. In such instances, oligos were designed to extend further into the deletion or insertion to enable accurate discrimination of the nucleotide change.

The microarray slides were coated with 3-Aminopropyl-trimethoxysilane plus 1,4-Phenylenedi-isothiocyanate (Asper Biotech, Ltd., Tartu, Estonia). The primers were diluted to 50 μM in 100 mM carbonate buffer (pH 9.0) and spotted onto the activated surface with BioRad VersArray (BioRad Laboratories, Hercules, Calif.). The slides were subsequently blocked with 1% ammonia solution and stored at 4° C. until needed. Prior to the APEX reactions, the slides were washed with 95° C. distilled water and 100 mM NaOH to reduce background fluorescence and to prevent re-hybridization of unbound oligonucleotides to the APEX slide.

Genomic and Synthetic Template Samples

45 bp synthetic templates were designed for each mutation site. 22 genomic DNA (gDNA) samples were collected having a total of 39 sequence variants represented on the microarray. Both genomic and synthetic templates that carry mutations present on the APEX array were used to validate each primer site.

The synthetic templates were designed according to the mutant gene sequence in both the forward and reverse direction. The design was optimized for melting temperature (MWG, Munich, Germany). Synthetic templates have been demonstrated to be a valid alternative for genomic patient DNA in the APEX assay (Schrijver, I., et al. Genotyping microarray for the detection of over 200 CFTR mutations in ethnically diverse populations. J Mol Diagn 2005; 7:375-387). For the gDNA samples, the SNHL genes of interest were amplified in pre-designed amplicons. The 50 μL PCR reaction was optimized, purified, and fragmented as previously described (Kurg, A., et al. Arrayed primer extension solid-phase four-color DNA resequencing and mutation detection technology. Genet Test 2000; 4:1-7). Prior to PCR amplification and subsequent microarray analysis, all gDNA samples were anonymized to ensure blind analysis. The results of the synthetic template validation were compared to the results obtained with the gDNA samples.

Arrayed Primer Extension (APEX) Reactions

The APEX mix consists of 32 μL of fragmented PCR product, 5U of Thermo Sequenase DNA polymerase (Amersham Pharmacia Biotech, Inc., Milwaukee, Wis.), 4 μL Thermo Sequenase reaction buffer (260 mM Tris-HCl, pH 9.5, 65 mM MgCl₂) (Amersham Pharmacia Biotech, Inc., Milwaukee, Wis.) and 1 μM final concentration of each fluorescently-labeled ddNTP: FL12-ddUTP, Cy3-ddCTP, Texas Red-ddATP, and Cy5-ddGTP (PerkinElmer Life Sciences, Wellesley, Mass. and custom synthesis by Amersham Pharmacia Biotech). The DNA was first denatured for ten minutes at 95° C. The enzyme and the dyes were then added to the DNA, and the mixture was applied to pre-warmed slides. The reaction was allowed to proceed for 10 minutes at 58° C., followed by one washing step with 0.3% Alconox (Alconox, Inc., White Plains, N.Y.) and two with distilled water for 90 seconds at 95° C. A small drop of antibleaching reagent (AntiFade SlowFade, Molecular Probes Europe BV, Leiden, The Netherlands) was applied to the slides before imaging.

Analysis

For each mutation site, forward and reverse oligos were spotted onto the APEX array. To reduce the possibility of false positive/negative results, the forward and reverse oligos were spotted in duplicate. Thus, every mutation site has four corresponding data points. This approach also enables distinction between homo- and heterozygosity at a mutation site. The array images were captured by a Genorama™ Quattrolmager 003 detector (Asper Biotech Ltd, Tartu, Estonia) at a resolution of 20 μm. This imager combines a total internal reflection fluorescence (TIRF) based excitation mechanism with a charge coupled device (CCD) camera (Tonisson N, Kurg A, Kaasik K, et al. Unravelling genetic data by arrayed primer extension. Clin Chem Lab Med 2000; 38:165-170). Sequence variants were identified using Genorama™ 3.0 genotyping software.

Results

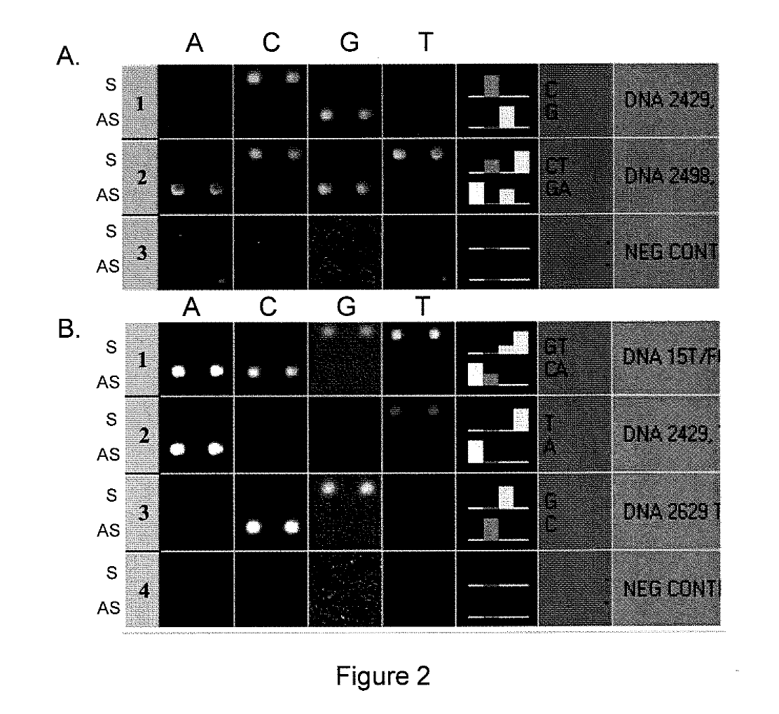

Representative results of the SNHL array are presented in FIGS. 2a and b. FIG. 2a demonstrates results of several individuals at the array spots for the R143W mutation in the GJB2 gene, which is common in individuals of African descent (Brobby, G. W., et al. Connexin 26 R143W mutation associated with recessive nonsyndromic sensorineural deafness in Africa. N Engl J Med 1998;338;548-550). In FIG. 2b, results from the grid position for the 35delG mutation are displayed. 35delG is the most commonly identified mutation in the GJB2 gene. This mutation causes a deletion of a single G in a string of 6, and is reliably identified by the SNHL array in both the forward and reverse direction.

gDNA Controls for Validation

22 gDNA patient samples with a total of 39 sequence variants were analyzed with the SNHL APEX microarray of the invention (see Table 2). Of these mutations, 37 were unique and two occurred twice. Of the 37 individual mutations, 18 were in the GJB2 gene, one in GJB6, and 18 in SLC26A4. The assay was shown to be highly specific, as no false positive mutations were detected. Specificity, however, is dependent on the labels on the dNTPs, which are not interchangeable in this assay and should be evaluated and optimized for each APEX array. We found one unexpected homozygous result for mutation IVS1+1G>A in SNHL-9 (Table 2). The homozygous result was reproduced twice more with the same PCR product upon APEX analysis. Upon re-amplifications of the original DNA for the purpose of another APEX analysis and a sequence analysis, both the DNA sequence and APEX analyses demonstrated heterozygosity. Therefore, we conclude that there was allele drop-out in the original PCR amplification (Piyamongkol, W., et al. Detailed investigation of factors influencing amplification efficiency and allele drop-out in single cell PCR: implications for preimplantation genetic diagnosis. Mol Hum Reprod 2003; 9:411-20.). This has occurred only once during development of the SNHL array of the invention. Allele drop-out is a very rare risk inherent to any method that uses PCR, and unrelated to the validation of microarray positions, or conditions of the APEX reaction itself. Taking into account this event, specificity (TN/TN+FP) was calculated as 97% (32/32+1). Because this false positive event was due to PCR amplification and not the APEX reaction itself, the specificity of the APEX assay is 100%.

In two instances, gDNA samples carried amino acid changes not present on the array (I203T in SNHL-1 and V653L in SNHL-22, as identified by sequencing). Sequence changes I203T and V653L were not included on the array because V653L is a known polymorphism and I203T is listed in the category of polymorphisms on the Connexin Deafness home page as “clinical significance not determined”. In both cases, a sequence change of the same amino acids is, in fact, located on the microarray (I203K and V653A, respectively). The two changes present in the gDNA samples did not result in false positive signals on the APEX array, which further underscores specificity of the APEX assay. Sensitivity of the assay was 100% and no false negatives were observed.

TABLE 2
Genomic DNA samples used for mutation evaluation on the APEX array.

| DNA No | Genotype GJB2 | Genotype GJB6 | Genotype SLC26A4 | Genotype, APEX | | Comments |
|---|---|---|---|---|---|---|
| SNHL-1 | V37I/I203T | na | na | GJB2 | V37I/WT | I203T of undetermined clinical |
| | | | | | | significance, not on the microarray. |
| | | | | | | No false detection of I203K |
| SNHL-2 | R16SW/V153I | na | ask lynn | GJB2 | R165W/WT | V153I is a polymorphism, |
| | | | | SLC26A4 | I490L/WT | not on the microarray. |
| SNHL-3 | G45E/ | na | na | GJB2 | G45E/ | All correct |
| | 176-191del16/ | | | | 176-191del16/ | |
| | Y136X | | | | Y136X | |
| SNHL-4 | V27I/E114G | na | na | GJB2 | V27I/E114G | Correct |
| SNHL-5 | V27I/WT | na | ne | GJB2 | V27I/WT | Correct |
| SNHL-6 | S139N/WT | na | na | GJB2 | S139N/WT | Correct |
| SNHL-7 | W24X/WT | na | na | GJB2 | W24X/WT | Correct |
| SNHL-8 | 167delT/WT | del 309 kb/WT | na | GJB2 | 167delT/WT | Correct |
| SNHL-9 | delE120/ | na | na | GJB2 | delE120 | Unexpected homozygosity. Allele |
| | IVS1 + 1G > A | | | | het/IVS1 + | drop out in PCR reaction. |
| | | | | | 1G > A hom | Consistent heterozygosity in |
| | | | | | | subsequent amlifications |
| SNHL-10 | 35delG/W77R | na | na | GJB2 | 35delG/W77R | Correct |
| SNHL-11 | H100P/S139N | na | na | GJB2 | H100P/S139N | Correct |
| SNHL-12 | R127H/WT | na | na | GJB2 | R127H/WT | Correct |
| SNHL-13 | na | na | M1T/L236P | SLC26A4 | M1T/L236P | Correct |
| SNHL-14 | na | na | G497S/E29Q | SLC26A4 | G497S/E29Q | Only the sense oligomers for |
| | | | | | | E29Q work well due to regional |
| | | | | | | genomic design complexity |
| SNHL-15 | na | na | L117F/WT | SLC26A4 | L117F/WT | Correct |
| SNHL-16 | na | na | L236P/S391N | SLC26A4 | L236P/WT | S391N is not on the microarray, |
| | | | | | | not in public databases. Novel. |
| SNHL-17 | na | na | 1147delC/G102R | SLC26A4 | 1147delC/WT | G102R is not on the microarray. |
| SNHL-18 | na | na | T416P/R409H | SLC26A4 | T416P/R409H | Correct |
| SNHL-19 | na | na | L445W/WT | SLC26A4 | L445W/WT | Correct |
| SNHL-20 | na | na | L236P/IVS14 + | SLC26A4 | L236P/IVS14 + | Correct |
| | | | 1G > A | | 1G > A | |
| SNHL-21 | na | na | L597S/WT | SLC26A4 | L597S/WT | Correct |
| SNHL-22 | G59A/WT | na | V609G/V653L | GJB2 | G59A/WT | V653L is a polymorphism not on |
| | | | | SLC26A4 | V609G/WT | the microarray, no false detection |
| | | | | | | of V653A. IVS13 − 14T > G is not |
| | | | | | | not in public databases, potentially |
| | | | | | | novel, not on the microarray. |

het = heterozygous; hom = homozygous

Synthetic Validation Controls

The detection of all mutations on the APEX array was validated with 45-mer synthetic oligomers. Their sequence was based on the wild type sequence, but with incorporation of the mutation to be identified. This approach allows validation of the APEX reaction and mutation detection with the Genorama™ Quattrolmager, but cannot assess optimization of the PCR steps which are required for gDNA samples, as synthetic templates are not amplified.

In these assays, sensitivity (TP/TP+FN) was 100% and specificity (TN/TN +FP) was 100%. The APEX reactions are entirely reproducible under the optimized testing conditions. Repeat testing at each site on the array for a heterozygous 35delG sample produced identical results in all eight repeats performed. The results described herein demonstrate that the APEX approach can detect numerous mutations that cause SNHL.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining whether a subject carries a HHL (Hereditary Hearing Loss) associated gene mutation, said method comprising:
(a) contacting an array comprising a plurality of distinct nucleic acid HHL gene mutation probes immobilized on a surface of a solid support with a nucleic acid sample from said subject to produce a sample contacted array;
(b) contacting said sample contacted array with a polymerase and at least two different distinguishably labeled dideoxynucleotides under primer extension conditions; and
(c) detecting the presence of any resultant terminally labeled nucleic acids immobilized on said substrate surface to determine whether said subject carries a HHL gene mutation.

2. The method according to claim 1, wherein said array comprises at least about 50 gene mutation probes for the mutations listed in Table 1.

3. The method according to claim 1, wherein said nucleic acid sample is an amplified genomic sample.

4. The method according to claim 3, wherein said amplified genomic sample is a fragmented amplified genomic sample.

5. The method according to claim 4, wherein said fragmented amplified genomic sample is an enzymatically fragmented sample.

6. The method according to claim 1, wherein said array comprises a plurality of pairs of HHL gene mutation probes, wherein each pair comprises a sense strand probe and an antisense strand probe.

7. The method according to claim 1, wherein said sample contacted array is contacted with four different distinguishably labeled ddNTPs.

8. The method according to claim 7, wherein said four different distinguishably labeled ddNTPs are ddATP, ddTTP, ddGTP and ddCTP.

9. The method according to claim 1, wherein said at least two dideoxynucleotides are labeled with fluorescent labels.

10. The method according to claim 9, wherein said detecting step comprises scanning said surface for said at least two different fluorescent labels.

11. The method according to claim 10, wherein said surface is scanned for four different fluorescent labels.

12. The method according to claim 1, wherein said method is a method for determining whether said subject is heterozygous for a HHL gene mutation.

13. The method according to claim 1, wherein said method is a method for determining whether said subject is homozygous for a HHL gene mutation.

14. An array comprising a plurality of at least about 50 distinct nucleic acid HHL gene mutation probes immobilized on a surface of a solid support.

15. The array according to claim 14, wherein said at least about 50 distinct gene mutation probes are for the mutations listed in Table 1.

16. The array according to claim 14, wherein said array comprises a plurality of pairs of HHL gene mutation probes, wherein each pair comprises a sense strand probe and an antisense strand probe.

17. The array according to claim 14, wherein said array comprises at least about 100 distinct nucleic acid HHL gene mutation probes.

18. The array according to claim 17, wherein said array comprises at least about 150 distinct nucleic acid HHL gene mutation probes.

19. (canceled)

20. A kit for use determining whether a subject carries a HHL (Hereditary Hearing Loss) associated gene mutation gene mutation, said kit comprising;
(a) an array comprising a plurality of at least about 50 distinct nucleic acid HHL gene mutation probes immobilized on a surface of a solid support; and
(b) at least two different distinguishably labeled dideoxynucleotides (ddNTPs).

21-26. (canceled)

27. A method of determining whether any of a plurality of subjects carry a HHL associated gene mutation, said method comprising:
(a) producing a plurality of nucleic acid samples from said plurality of subjects, wherein each of said plurality of nucleic acid samples corresponds to one of said plurality of subjects;
(b) contacting each of said plurality of nucleic acid samples with an array comprising a plurality of distinct nucleic acid HHL gene mutation probes immobilized on a surface of a solid support to produce a plurality of sample contacted arrays;
(c) contacting each of said plurality of sample contacted arrays with a polymerase and at least two different distinguishably labeled dideoxynucleotides under primer extension conditions; and
(d) detecting the presence of any resultant terminally labeled nucleic acids immobilized on said substrate surface to determine whether any of said plurality of subjects carry a HHL gene mutation.

28. The method according to claim 27, wherein the accuracy of said method is about 90% or greater.

29-30. (canceled)
